(19) United States
(12) Patent Application Publication
(10) Pub. No.: US 2015/0221232 A1
(43) Pub. Date: Aug. 6, 2015

(54) SYSTEM AND METHODS FOR CREDITING PHYSICAL ACTIVITY PERFORMED BY A USER

(71) Applicant: Go Vivo Ltd., Ramat Gan (IL)

(72) Inventors: Ilan ZIV, Tel Aviv (IL); Elad RAZ, Ramat Gan (IL)

(73) Assignee: Go Vivo Ltd., Ramat Gan (IL)

(21) Appl. No.: 14/587,425

(22) Filed: Dec. 31, 2014

Related U.S. Application Data
(63) Continuation of application No. PCT/IL2013/050526, filed on Jun. 19, 2013.
(60) Provisional application No. 61/667,520, filed on Jul. 3, 2012.

(51) Int. Cl. G09B 19/00; A61B 5/00; A61B 5/11

(52) U.S. Cl. CPC G09B 19/00; A61B 5/1118; A61B 5/4833; A61B 5/7465

(57) ABSTRACT

A method and system for incentivizing a user of a user device to perform physical activity are provided. The method comprises receiving at least one signal respective of a physical activity performed by the user, wherein the at least one signal is received from at least one sensor integrated in a user device; generating at least one virtual good respective of the received at least one signal; and sending the at least one virtual good to at least one web source, wherein the virtual good can be redeemed by the user through the at least one web source.

### CROSS REFERENCE TO RELATED APPLICATIONS

This application is a continuation of PCT Application No. PCT/IL-2013/050526 filed on Jun. 19, 2013, now pending, which claims the benefit of U.S. provisional patent application No. 61/667,520 filed on Jul. 3, 2012. All of the above-referenced applications are hereby incorporated by reference for all that they contain.

### TECHNICAL FIELD

This disclosure generally relate to an apparatus for tracking a user's activity and, more particularly, to a system and methods for tracking physical activity performed by a user and generating virtual goods respective of the physical activity.

### BACKGROUND

The vast growth of content available over the Internet has led to a significant increase in the amount of time people interact with a computer. This phenomenon is especially common among children, who are more inclined to stay indoors and play electronic games rather than participating in outdoor physical activities.

Researches show that, during the 1970s and 1980s, children participated in over two hours of play outside each weekday and a further nine hours of play outside on weekends. These days, the time children spend outside playing is estimated at no more than an hour each weekday, and less than five hours on weekends.

This decrease in the time people and, in particular, children, spend performing physical activities outside is concerning because outdoor physical activity not only helps children to stay active and healthy, but also assists in socializing with friends.

It would therefore be advantageous to provide a solution that would incentive users to participate in outdoor physical activities by granting electronic rewards for participation in such activities.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a schematic diagram of a system for tracking physical activity performed by a user according to an embodiment;

FIG. 2 is a schematic diagram of an apparatus for tracking physical activity performed by a user according to an embodiment;

FIG. 3 is an isometric view of an apparatus for tracking physical activity performed by a user according to an embodiment;

FIG. 4 is a flowchart describing the operation of a method for tracking physical activity performed by a user according to an embodiment; and

FIG. 5 is a flowchart describing the operation of generating virtual goods respective of physical activity performed by a user according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

By a way of example for the disclosed embodiments, an electronic system provides incentives to users to participate in physical activities. The system is configured to track physical activities performed by a user and provides the user with virtual rewards based on the physical activities. Signals respective of physical activity performed by the user are collected by one or more sensors integrated in a portable device. The portable device may be, for example, a watch or a bracelet. The signals are then analyzed and converted to virtual goods of which the user can use through one or more web sources.

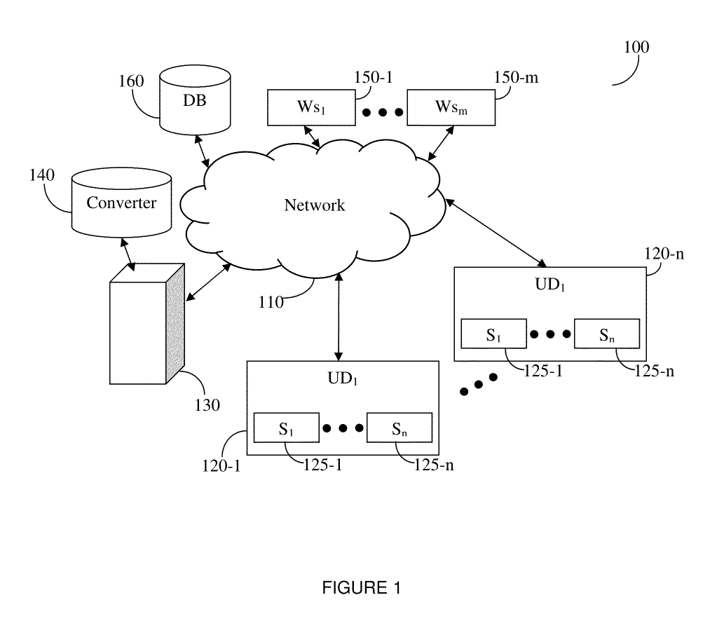

FIG. 1 is an exemplary and non-limiting schematic diagram of a system 100 for tracking physical activity performed by a user according to an embodiment. A network 110 is used to communicate between different parts of the system 100. The network 110 may be the Internet, the world-wide-web (WWW), a local area network (LAN), a wide area network (WAN), a metro area network (MAN), and other networks capable of enabling communication between the elements of the system 100. Further connected to the network 110 are one or more user devices 120-1 through 120-n (hereinafter referred to collectively as user devices 120 or individually as a user device 120, merely for simplicity purposes). The user device 120 may be, for example, a mobile phone, a smart phone, a tablet computer, a portable device, and other kinds of wired and mobile appliances as further described herein below. A portable device may be, for example, a bracelet, a watch, a tag, a hair accessory, and so on, equipped with a processing unit.

The user device 120 comprises one or more sensors 125-1 through 125-n that are configured to collect one or more signals related to physical activities performed by the user. Physical activity may be, for example, running, walking, jumping, crawling, climbing, rowing, exercising, and so on. Each one of the sensors 125-1 through 125-n may be, but is not limited to, a temperature sensor, a sound sensor, an ultrasound sensor, an accelerometer, a heartbeat sensor, a dampness sensor, a blood pressure sensor, a sweat detector, a camera, and the like. According to one embodiment, upon identification that the user device 120 crosses a certain preconfigured threshold, a notification to the user is generated. The notification assists in ensuring that the user will not reach an over training situation.

A server 130 is further connected to the network 110, configured to send and receive data to and from the user devices 120. The system 100 also includes a converter 140 that is connected to the server 130. The converter 140 is configured to convert signals related to physical activity collected by the one or more sensors of the user device 110 to virtual goods based on one or more predetermined conversion rates. Virtual goods refers to any type of compensation, virtual or physical, which the user is able to redeem through one or more web sources as further described herein below. According to one embodiment, the converter 140 is integrated in the user device 120. According to another embodiment, the converter 140 is integrated in the server 130. According to yet another embodiment, the conversion process may be partitioned such that part of the conversion is performed on the user device 120 and part of the conversion is performed on the server 130.

It should be noted that each of the server 130, the user device 120, and the converter 140 typically comprises a processing unit, such as a processor (not shown) that is coupled to a memory (not shown). The memory contains instructions that can be executed by the processor. The server 130 also includes an interface (not shown) to the network 110.

Further connected to the network 110 are one or more web sources 150-1 through 150-m, in which the virtual goods may be used. Virtual goods may be used by, for example, providing a power up for an in-game character, advancing a level, purchasing a physical item, purchasing an in-game item, and so on.

According to one embodiment, a user selection of one or more web sources 150 in which the virtual goods are to be used is received from a user device 120. The web sources may be, for example: virtual world web sites, shopping websites, and so on. The virtual goods may be treated by any of the web sources 150 as virtual credits for trade through or with any of the web sources 150. According to one embodiment, the converter 140 converts the collected signals to one or more numeric values. Typically, the numerical values associated with collected signals increase accordingly as the amount of physical activity demonstrated via the collected signals increases.

The one or more numeric values are sent to a web source of the one or more web source 150, for example the web source 150-1. The selected web source 150-1 analyzes the numeric values and generates virtual goods respective thereto. The system 100 may further comprise a database 160 that is configured to store data related to the physical activity performed by users and the virtual goods received respective thereto.

According to the embodiments disclosed herein, the user device 120 is carried by a user. The plurality of sensors 125 collects one or more signals related to the physical activity performed by the user such as, for example, running pace, walking pace, heart-rate or heart-bits, blood pressure, level of sweat, distance, skin color changes, and so on. The signals are sent from the user device 120 to the server 130 over the network 110. The connection between the user device 120 and the server 130 may be encrypted or sent, for example, via a virtual private network (VPN), thereby preventing forgery of the user devices 120.

According to another embodiment, in cases where the user device 120 is not connected to the network 110, a manual connection through a proxy device is required in order to transfer the signals to a device that enables sending the signals over the network 110 to the server 130. The connection to the proxy device may be by a cable, a Universal Serial Bus (USB) connection, an Ethernet connection, and any other wired or wireless connection such as, Wi-Fi, Bluetooth, and so on.

Thereafter, the server 130 is further configured to analyze the collected signals. The analysis may include extracting personal information related to the user that may be used for determination of the virtual value of the signals. Personal information may include information related to the user's age, physical condition, physical goals, gender, education level, and so on. Respective of the personal information, the virtual value of the physical activity is determined. As an example, physical activity performed by a young athlete converts to virtual goods based on a certain conversion rate while the same activity performed by an elderly woman converts to virtual goods based on a different conversion rate. According to one embodiment, the conversion rates may be determined by the operators of each of the web sources 150 and, therefore, each web source may have a different conversion rate. Furthermore, web source operators may determine that certain activities may not be considered eligible for virtual goods. The signals are forwarded to the converter 140 by the server 130. The converter 140 converts the signals to virtual goods and forwards the virtual goods to the server 130. The server 130 sends the virtual goods to the designated one or more web sources 150.

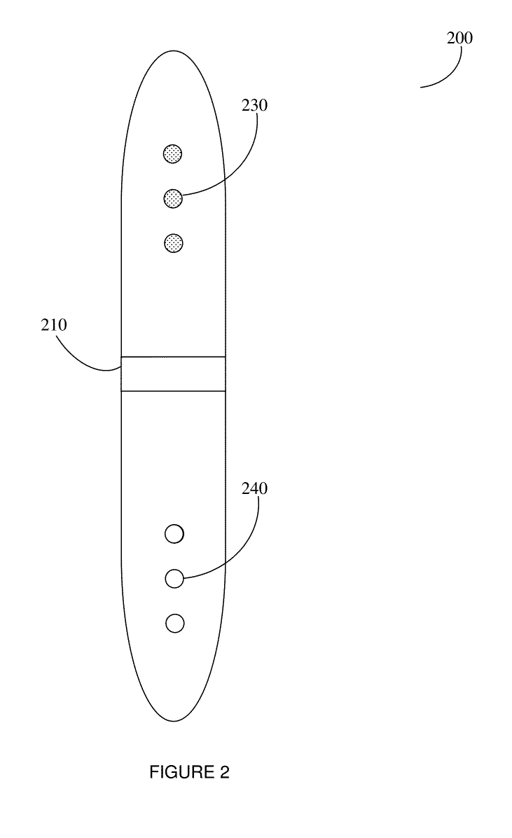

FIG. 2 is an exemplary and non-limiting schematic diagram of a wrist bracelet 200 for tracking physical activity performed by a user according to an embodiment. The wrist bracelet 200 comprises a module 210 that is configured to track the user's physical activity in real-time and convert it to virtual goods. The module is configured to generate a notification indicating the amount of virtual goods accumulated to the user in real-time. The wrist bracelet 200 may be adjusted respective of the size of the user's wrist. The adjustment may be achieved by a line of several pins 230 located on a first strap of the bracelet 200 which correspond to several holes 240 located on a second strap of the bracelet 200.

The module 210 includes a plurality of sensors (e.g., the sensors 125) for collection of one or more signals related to physical activity performed by the user as further described hereinabove with respect to FIG. 1. The module 210 further includes an interface to the network 210 through which the signals may be sent to a server (e.g., the server 130) for further processing. According to another embodiment, the module 210 is further configured to process the signals, to convert the signals to virtual goods locally, and to send the virtual goods directly to one or more web sources (e.g., the web sources 150). According to one embodiment, the bracelet 200 may further enable sharing of virtual goods with other devices through a network (e.g., the network 110). The bracelet 200 may further comprise an area over which advertisements may be shown. According to yet another embodiment, the bracelet may include an identification unit (not shown) configured to verify the identity of the user while performing physical activity via, for example, fingerprint identification.

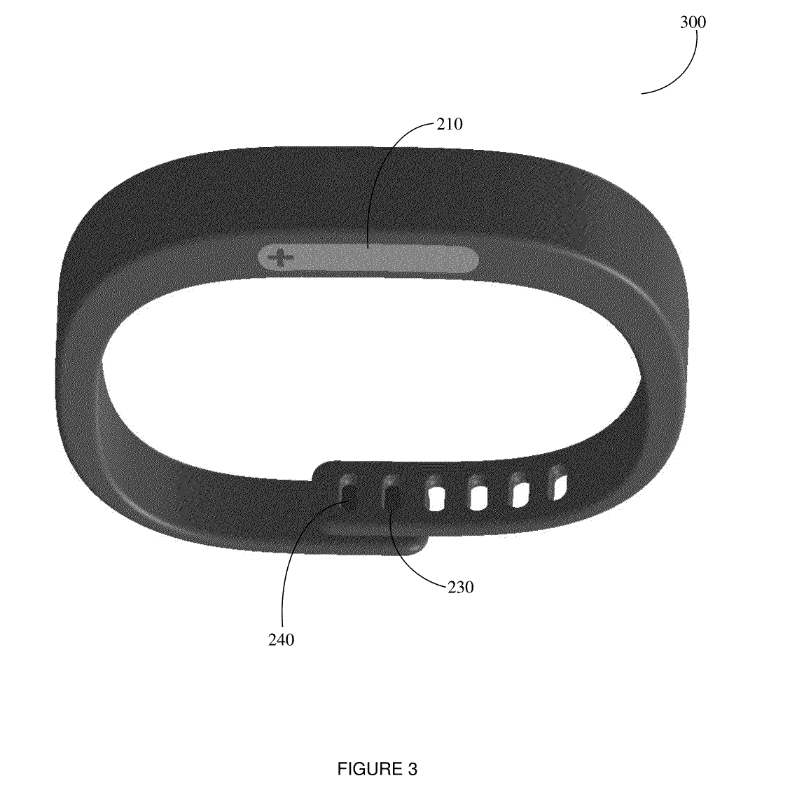

FIG. 3 is an exemplary and non-limiting isometric view of a wrist bracelet 300 for tracking physical activity performed by a user according to the embodiment described herein above with respect to FIG. 2.

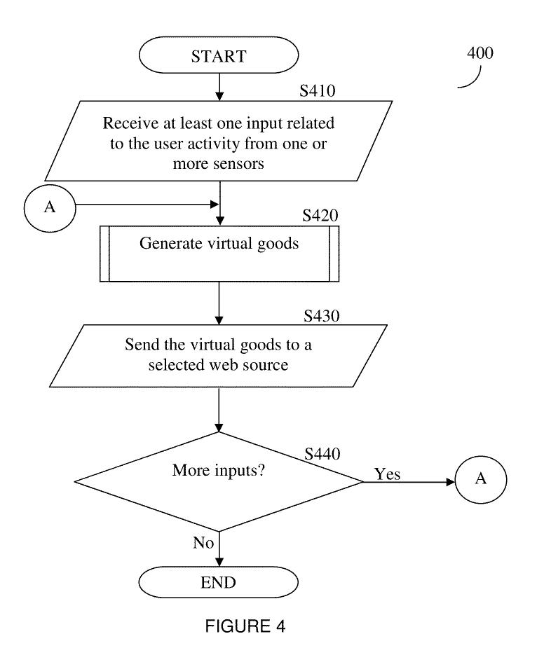

FIG. 4 is an exemplary and non-limiting flowchart 400 illustrating a method for tracking physical activity performed by a user according to an embodiment. In S410, at least one input related to the user's activity is received. Such an input may be, but is not limited to, a signal collected by one or more sensors (e.g., the sensors 125).

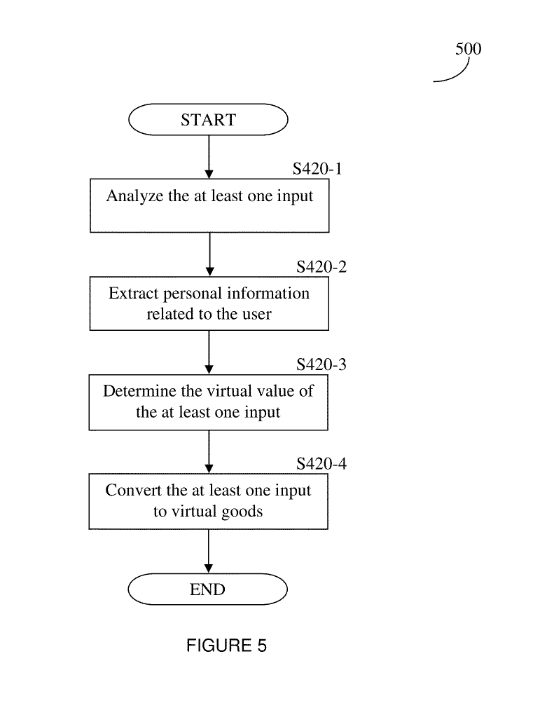

In S420, respective of the at least one input, virtual goods are generated. Generation of virtual goods respective of the input is further described herein below with respect to FIG. 5. According to one embodiment, in order to achieve a power consuming operation, the collected signals may be accumulated in a user device (e.g., the user device 120) and generation of virtual goods is performed upon reaching a predetermined threshold. In S430, the generated virtual goods are sent to a selected one or more web sources (e.g., the web sources 150).

In S440, it is checked whether additional inputs have been received and, if so, execution continues with S420; otherwise, execution terminates. According to another embodiment, metadata related to either or both of the physical activity performed by the user and the virtual goods is stored in a data warehouse (e.g., the database 160) for further use. The metadata related to the physical activity maybe a location pointer respective of the physical activity, a type of the physical activity, identifiers related to the user, a combination thereof, etc. Metadata related to the virtual goods may include, for example, past selection(s) of virtual goods.

FIG. 5 is an exemplary and non-limiting flowchart S420 illustrating a method of generating virtual goods respective of at least one input related to physical activity performed by a user according to an embodiment. In S420-1, the at least one input is analyzed. In one embodiment, the analysis may include, but is not limited to, converting raw data (i.e., electrical signals) into scores. In an embodiment, raw data from different sensors may be converted into different scores. As an example, running may be converted to a certain score and jumping may be converted to a different score.

In optional S420-2, personal information related to the user is extracted. Personal information may include information related to the user's age, physical condition, physical goals, gender, education level, and so on. In S420-3, the virtual value of the at least one input is determined. The virtual value of an input represents the amount or significance of virtual goods awarded to the user. The determination is achieved by analyzing at least one of: the at least one input, and the personal information related to the user. In S420-4, the at least one input is converted to a virtual good and execution terminates.

It should be clear to one of ordinary skill in the art that the process of generating virtual goods respective of at least one input related to physical activity performed by the user may be executed by any of: a user device (e.g., the user device 120), or a server (e.g., the server 130).

The disclosed embodiments may be implemented as hardware, firmware, software, or any combination thereof. Moreover, the software is preferably implemented as an application program tangibly embodied on a program storage unit or tangible computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit. All or some of the servers maybe combined into one or more integrated servers. Furthermore, a non-transitory computer readable medium is any computer readable medium except for a transitory propagating signal. The display segments and mini-display segments may be shown on a display area that can be a browser or another other appropriate application, either generic or tailored for the purposes described in detail hereinabove.

All examples and conditional language recited herein are intended for pedagogical purposes to aid the reader in understanding the disclosed embodiments and the concepts contributed in order to further the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for incentivizing a user of a user device to perform physical activity, comprising:
receiving at least one signal respective of a physical activity performed by the user, wherein the at least one signal is received from at least one sensor integrated in the user device;
generating at least one virtual good respective of the received at least one signal; and
sending the at least one virtual good to at least one web source, wherein the virtual good can be redeemed by the user through the at least one web source.

2. The method of claim 1, further comprising:
analyzing the at least one signal to at least determine a type of the physical activity;
determining a virtual value associated with the at least one signal respective of the physical activity; and
converting the virtual value into the at least one virtual good.

3. The method of claim 1, further comprising:
extracting personal information related to the user.

4. The method of claim 1, wherein the user device is any one of: a mobile phone, a smart phone, a tablet computer, and a portable device.

5. The method of claim 4, wherein the portable device is any one of: a bracelet, a watch, a tag, and a hair accessory.

6. The method of claim 1, wherein the sensor is any one of: a temperature sensor, a sound sensor, an ultrasound sensor, an accelerometer, a heartbeat sensor, a dampness sensor, a blood pressure sensor, a sweat detector, and a camera.

7. The method of claim 1, further comprising:
generating a real-time notification upon identification that a signal of the at least one signal has crossed a predetermined threshold.

8. The method of claim 1, further comprising:
receiving a selection of the at least one web source to redeem the virtual goods from the user device.

9. The method of claim 1, further comprising:
collecting metadata related to the physical activity of the user.

10. The method of claim 1, further comprising:
verifying an identity of the user to determine that a designated user actually performs the physical activity.

11. A non-transitory computer readable medium having stored thereon instructions for causing one or more processing units to execute the method according to claim 1.

12. A system for incentivizing a user of a user device to perform physical activity, comprising:
an interface to a network for receiving and sending data over the network;
a processing unit; and
a memory coupled to the processing unit, the memory containing instructions that, when executed by the processing unit, configure the system to:
receive at least one signal respective of a physical activity performed by the user, wherein the at least one signal is received from at least one sensor integrated in the user device;
generate at least one virtual good respective of the received at least one signal; and
send the at least one virtual good to at least one web source, wherein the virtual good can be redeemed by the user through the at least one web source.

13. The system of claim 12, wherein the system is further configured to:
analyze the at least one signal to at least determine a type of the physical activity;
determine a virtual value associated with the at least one signal respective of the physical activity; and
convert the virtual value into the at least one virtual good.

14. The system of claim 12, further comprising:
extracting personal information related to the user.

15. The system of claim 12, wherein the user device is any one of: a mobile phone, a smart phone, a tablet computer, and a portable device.

16. The system of claim 15, wherein the portable device is any one of: a bracelet, a watch, a tag, and a hair accessory.

17. The system of claim 12, wherein the sensor is any one of: a temperature sensor, a sound sensor, an ultrasound sensor, an accelerometer, a heartbeat sensor, a dampness sensor, a blood pressure sensor, a sweat detector, and a camera.

18. The system of claim 12, wherein the system is further configured to:
generate a real-time notification upon identification that a signal of the at least one signal has crossed a predetermined threshold.

19. The system of claim 12, wherein the system is further configured to:
receive a selection of the at least one web source to redeem the virtual goods from the user device.

20. The system of claim 12, wherein the system is further configured to:
collect metadata related to the physical activity of the user.

21. The system of claim 12, wherein the system is further configured to:
verifying an identity of the user to determine that a designated user actually performs the physical activity.

22. An apparatus for incentivizing a user of a user device to perform physical activity, comprising:
a plurality of sensors for collecting at least one signal respective of a physical activity performed by the user;
a network interface for sending the at least one signal over the network, wherein at least one virtual good is generated respective of the at least one collected signal and sent to at least one web source, wherein the virtual good can be redeemed by the user through the at least one web source.

23. The apparatus of claim 23, further comprising:
a converter for converting the at least one signal into the at least one virtual good, wherein the at least one virtual good may be sent over the network via the network interface.

24. The apparatus of claim 23, further comprising:
an identification unit configured to receive an identifying input from a user and to verify an identity of the user respective of the identifying input.

25. The apparatus of claim 15, wherein the portable device is any one of: a bracelet, a watch, a tag, and a hair accessory.
